SEQUENCE LISTING (5 sequences)

SEQ ID NO: 1 - DNA, 20 bp, Artificial
  PCR primer
cacaacagaagccaggaacc

SEQ ID NO: 2 - DNA, 20 bp, Artificial
  PCR primer
gaggggacgacgacagtatc

SEQ ID NO: 3 - DNA, 20 bp, Artificial
  PCR primer
caacggagccaggggaggtg

SEQ ID NO: 4 - DNA, 20 bp, Artificial
  PCR primer
cggcgacacagaaccaacaa

SEQ ID NO: 5 - DNA, 20 bp, Artificial
  PCR primer
gaggggacgacgacagtatc

(19) United States
(12) Patent Application Publication
(10) Pub. No.: US 2005/0177885 A1
(43) Pub. Date: Aug. 11, 2005

(54) CCN1 transgenic animals

(76) Inventor: Lester F. Lau, Chicago, IL (US)

Correspondence Address: HOWREY SIMON ARNOLD & WHITE LLP;Attention: Patent Administrator, Box No. 34, 1299 Pennsylvania Avenue, N.W., Wahington, DC, 20004-2402, US

(21) Appl. No.: 10/774,706

(22) Filed: Feb. 9, 2004

(51) Int. Cl. A01K 67/027

(52) U.S. Cl. 800/18

(57) ABSTRACT

The invention describes transgenic animals with a heterozygous mutation of the CCN1 gene and animal models for screening treatments for atrioventricular septal defects.

### BACKGROUND OF THE INVENTION

1. Field of the Invention

The present invention concerns novel CCN1-transgenic mammals and their use as a model to test therapies for treating or preventing atrioventricular septal defects (AVSDs).

2. Description of Related Art

a. AVSD

Atrioventricular septal defects (AVSDs), also referred to as endocardial cushion or atrioventricular canal defects, make up 5% of congenital heart diseases (CHDs). AVSDs include a range of CHDs characterized by varying degrees of incomplete development of the inferior portion of the atrial septum (the muscular wall separating an atrium on the left side of the heart from an atrium on the right), the inflow portion of the ventricular septum (the muscular wall dividing a ventricle on the left side of the heart from a ventricle on the right), and atrioventricular valves.

The six common features seen in atrioventricular septal defects are as follows: sprung atriventricular junction; inlet-outlet disproportion; absence of the muscular atrioventricular septum; abnormal papillary muscle distribution; abnormal atrioventricular valve leaflet configuration; and cleft in the left atrioventricular valve. Unlike the normal heart where the atrioventricular junction has a figure-of-eight appearance, in all cases of atrioventricular septal defect, the junction is sprung. This appearance is independent of the presence or absence of an interatrial or interventricular communication.

Inlet-outlet disproportion is common to all forms of atrioventricular septal defect and is a direct result of the sprung atrioventricular junction. The sprung atrioventricular junction results in the aorta (in ventriculoarterial concordance) lying in an unwedged position above the atriventricular junction, such that the normal 1:1 ratio of the inlet to outlet is abnormal. This component of an atrioventricular septal defect plays an important role in the genesis of left ventricular outflow tract obstruction that may complicate this lesion.

In all cases of atrioventricular septal defects, the muscular atrioventricular septum is absent. The left ventricular pappilary muscles in atriventricular septal defect are rotated in a counter-clockwise direction such that the mural or posterior leaflet is shorter than that observed in a normal heart. This rotation of the papillary muscles is an important component of this lesion, as their new location supports the bridging leaflets, and hence, explains the direction that the so-called cleft points in atrioventricular septal defect. In the normal heart, there is a smaller anterior or aortal leaflet of the mitral valve in fibrous continuity with the noncoronary cusp of the aortic valve, with a broad posterior leaflet with several scallops. The cleft in antrioventricular septal defect in fact represents a commissure between the superior and inferior bridging leaflets.

While all atrioventricular septal defects have a sprung atrioventricular junction, there may be a partitioned or common atrioventricular valve orifice. This depends on the presence or absence of a connecting tongue of tissue linking the superior and inferior bridging leaflets.

Atrioventricular septal defects are often divided into complete (CAVSD) and partial (PAVSD), depending on the presence or absence of a common atrioventricular valve. CAVSD is characterized by a common valve orifice and confluent atrial and ventricular septal defects. PAVSD is characterized by separate right and left atrioventricular orifices and the anatomical potential only for atrial shunting. Malformations may also rarely exist with the potential for shunting exclusively at ventricular level. Irrespective of these variations, the left atrioventricular valve in all atrioventricular septal defects has a characteristic three-leaflet arrangement. The left ventricular outflow tract is always narrow and is particularly susceptible to obstruction. Other associated malformations include additional muscular ventricular septal defects, valvar pulmonary stenosis or more complex lesions like Fallot's tetralogy or double outlet right ventricle.

In patients with complete atrioventricular septal defect (CAVSD) the deficiency in the inlet portion of the ventricular septum is greater than that in those with partial defects (PAVSD). Patients with CAVSD usually become symptomatic within the first year of life. In these patients, pulmonary vascular disease develops within a few months. These patients also suffer from poor feeding and thus, early surgery, within the first year of life, is recommended. Patients with PAVD may be asymptomatic. However, they often have cardiac murmurs, which are discovered at routine medical checks. The indication for operation in patients with PAVSD is elective and performed in the first decade of life (Meisner, 1998).

Atrioventricular defects can be detected by Doppler echocardiography. At characterization, the left atrium is easily entered in the majority of patients. Wayward behavior of the catheter, which enters all chambers easily and moves from one to another with little manipulation, is a clue to the presence of a common valve orifice. A left-to-right shunt is usually detected first at atrial level, but subsequent rises in oxygen saturation in the right ventricle or pulmonary trunk do not differentiate the various forms. More important is the presence or the absence of pulmonary hypertension. The pulmonary arterial pressure is usually normal when shunting is only at atrial level but high in those with common valves. The left atrial pressure provides little indication concerning the presence or severity of regurgitation through the left atrioventricular valve. It will be elevated only if the inter-atrial communication is restrictive.

Three factors have a major effect on patient's longevity: the presence and severity or regulation through the left atrioventricular valve; the development of pulmonary vascular disease (which is usually related to the presence and size of any interventricular communication); and the integrity of the conduction tissues.

AVSDs are commonly present in Down's syndrome (trisomy 21), and approximately 40% of patients with this syndrome have AVSDs, often in association with a cleft mitral valve. The trisomy 16 mouse has been used as a model of Down syndrome. These mice have a well-described cardiac phenotype that includes AVSDs. Using quantitative morphometry, it has been shown that the trisomy 16 embryos have delayed development of mesenchymal cells in the endocardial cushions with fewer mesenchymal cells been present in the cushions (Gelb, 1997).

Mendelian inheritance of isolated AVSDs occasionally occurs in the absence of Down syndrome. These familial AVDSs can be transmitted in an autosomal dominant pattern, and linkage analysis excluded loci on chromosome 21 (Cousineau et al., 1994).

Recent discoveries with molecular genetic studies have shown that single gene or single locus abnormalities account for many of the heart lesions or syndromes with CHDs. As mentioned above, AVSDs are most commonly associated with trisomy 21 (Down syndrome) in humans. Other chromosome abnormalities associated with AVSDs include trisomy 13, trisomy 18, deletion 3p25, and deletion 8p2 (see, e.g., Markwald et al. [2000]). In addition, there are a number of extended families with multiple individuals with AVSD where there are no chromosome abnormalities. These reports indicated that these AVSDs were resulting from a major susceptibility gene (see Disegni et al. [1985] and Kumar et al. [1994]). By using DNA pooling and shared segment analysis, a genetic locus for an AVSD susceptibility gene on chromosome 1 (1p21-p31) was identified from a four-generation kindred (see Sheffield et al. [1997]). Since the current critical interval is still large, ˜12 cM, a specific gene responsible for AVSD has yet to be defined.

Therefore, there exists a need to identify the genetic cause of AVSD. The identification of the genetic cause of AVSD could lead to the development of genetic screens for AVSD. The identification of the genetic cause of AVSD could also lead to the development of an animal model for AVSD, which could be used for testing therapeutics and treatments for AVSD and other associated congenital heart diseases.

b. CCN1

Among the many genes present within the 12 cM interval believed to contain an AVSD susceptibility gene is the gene CCN1/Cyr61, which has been mapped to human chromosome 1p22-p31 (see Jay et al. [1997]). CCN1 is a member of the CCN protein family, which includes Cyr61/CCN1, CTGF/CCN2 (connective tissue growth factor), NOV/CCN3 (nephroblastoma overexpressed), Elm-1/WISP-1/CCN4, Cop-1/WISP-2/CCN5, and WISP-3/CCN6. The CCN proteins share four modular domains with sequence similarities to insulin-like growth factor-binding proteins, von Willebrand factor, thrombospondin, and cysteine knots also found in growth factors for dimerization (see Lau & Lam [1999]).

CCN1 is a growth factor-inducible immediate-early gene initially identified in serum-stimulated mouse fibroblasts (Lau & Nathans [1985]). CCN1 has been shown to mediate cell adhesion, stimulate cell migration, and enhance growth factor-stimulated DNA synthesis in both fibroblasts and endothelial cells in culture (see Kireeva et al. and Babic et al. [1998]). CCN1 regulates the expression of genes involved in angiogenesis and matrix remodeling, including VEGF-A, VEGF-C, type I collagen, MMP1, and MMP3 (see Chen et al. [2001]).

c. CCN1 Knock-Out Mice

The mouse CCN1 gene has been insertionally inactivated (i.e., knocked out) in vivo by targeted gene disruption (Lau et al., WO 01/55210). Heterozygous mice (CCN1^{+/-}) appeared normal and did not exhibit any apparent phenotype. The CCN1^{-/-} homozygous mice, however, exhibited severe vascular defects and apparent neuronal defects as well. Most of the CCN1^{-/-} mice died in utero, starting from E10.5 through parturition, with most embryos dying around E13.5. The CCN1^{-/-} homozygous mice displayed a wide spectrum of developmental defects and phenotypes at the time of embryonic death.

### BRIEF SUMMARY OF THE INVENTION

The present invention relates to a transgenic mouse with a genome comprising a heterozygous disruption of the CCN1 gene. The mouse may be predisposed to atrioventricular septal defects. The mouse may also have atrioventricular septal defects. The mouse may also be an embryo. The mouse may also be in contact with a suspected modulator of effects associated with congenital heart disease.

The present invention also relates to a homogeneous population of transgenic mice with a genome comprising a heterozygous disruption of the CCN1 gene. The population of mice may be predisposed to having atrioventricular septal defects. The population of mice may have atrioventricular septal defects.

The present invention also relates to a method producing a mouse with atrioventricular septal defects, comprising producing a transgenic mouse whose genome comprises a heterozygous disruption of the CCN1 gene; testing the transgenic mouse for the presence of a phenotype associated with atrioventricular septal defects; and, isolating a transgenic mouse that has a phenotype associated with atrioventricular septal defects. The mouse may be an embryo.

The present invention also relates to a method of isolating a mouse with atrioventricular septal defects, comprising testing a transgenic mouse whose genome comprises a heterozygous disruption of the CCN1 gene for the presence of a phenotype associated with atrioventricular septal defects; and, isolating a transgenic mouse that has a phenotype associated with atrioventricular septal defects. The mouse may be an embryo.

The present invention also relates to a method of identifying a mouse with atrioventricular septal defects, comprising testing a transgenic mouse whose genome comprises a heterozygous disruption of the CCN1 gene for the presence of a phenotype associated with atrioventricular septal defects. The mouse may be an embryo.

The present invention also relates to a method of identifying a modulator of symptoms associated with atrioventricular septal defects, comprising contacting a transgenic mouse whose genome comprises a heterozygous disruption of the CCN1 gene with a suspected modulator, and measuring a phenotype associated with atrioventricular septal defects, whereby a modulator is identified by altering the phenotype in comparison to a control. The mouse may be an embryo.

The present invention also relates to a method of identifying an animal that is predisposed to atrioventricular septal defects, comprising detecting the presence of an alteration in one or more alleles of the CCN1 gene in a sample comprising DNA isolated from said animal. The mouse may be an embryo.

### BRIEF DESCRIPTION OF THE DRAWINGS

FIG. 1 shows the expression of CCN1 in the embryonic hearts. Panel A: Whole-mount X-gal staining of a E10.5 CCN^{+/-} embryos with arrows pointing to the heart with intensive staining. Panel B: A heart dissected from E10.5 embryo with arrows pointing to the major expression area at truncus arteriosus. Panel C: Arrows pointing to expression at both the atrioventricular cushion tissue and interventricular septum (IVS). Panel D: During the onset of closing IVS at E12.5, X-gal staining is highlighted at the junction between the septum muscular component and the membranous mesenchyme (open arrow) and the valvular structure (solid arrow) with staining also observed at the upper part of cushion tissue where fusion with septum primum occurs. Panel E: Whole-heart staining of E13.5 embryo showing that expression is localized to AV cannel (open arrow) and valves (solid arrow). Panel F: Section of E13.5 heart demonstrating intense staining at mitral valve leaflets. Bars: 200 μm.

FIG. 2 shows histological analysis of E14.5 embryo hearts. Panel A: Wild-type heart demonstrates septation of atrium and ventricle with arrows pointing to complete closing of IVS. Panel B: Four-chamber structure is formed with the AV valves being developed (arrows). Panel C: VSD (arrowhead) and ASD (arrow) were observed in CCN1^{+/-} hearts. Panel D: Isolated cleft mitral valves (arrow) was found in some of the CCN1^{+/-} hearts, while the tricuspid valves (arrowhead) appeared normal. More severe phenotype detected in CCN1^{-/-} hearts. The CCN1^{-/-} hearts show ASD (arrow in e), and VSD (arrowhead in e). The under-development of the cushion tissue (arrow in f) causes failure of AV valve formation.

FIG. 3 shows histological analysis of postnatal atrium septation on one-day-old pups. In wild-type, the ostium secundum (arrow in a) was closed by the septum secundum. As the septum secundum (arrow in b) never reaches the cushion tissue, the space in between was closed by the septum primum (arrowhead in b). Panel C:. In defective CCN1^{+/-} pups, the communication between two chambers of atrium remains. The ostium primum (arrow) in the lower part of septum primum (arrowhead) was not closed by the fusion between cushion tissue and septum primum. As a result, excessive blood can be seen trapped in the atrium.

### DETAILED DESCRIPTION OF THE INVENTION

As used herein unless the context dictates otherwise, italicizing the name of a gene shall indicate the gene, in contrast to its encoded protein product which is indicated by the name of the gene in the absence of any underscoring or italicizing. For example, “CCN1” shall refer to the protein product of the “CCN1” gene.

As discussed above, heterozygous CCN1^{+/-} transgenic mice appeared to be normal whereas the majority of homozygous CCN1^{+/-} transgenic mice died in utero. As described more fully in the examples herein, heterozygous CCN1^{+/-} transgenic mice are surprisingly predisposed to congenital heart disease. Heterozygous CCN1^{+/-} mice display a phenotype similar to human AVSD with incomplete penetrance and variable expressivity from isolated atrial or ventricular septal defects, cleft mitral valves to complete AVSD.

The CCN1^{+/-} mutation is a rare mouse model displaying heterozygous cardiac phenotype with single gene mutation, in accordance with the autosomal dominant inheritance observed in most the human CHDs. Our results demonstrate a novel pathogenic mechanism of AVSDs and provide an animal model for further studies on this type of human CHDs.

The present invention relates to a transgenic animal whose genome comprises a heterozygous mutation of the CCN1 gene. The transgenic animal may be predisposed to AVSDs. The transgenic animal may also have AVSDs. The present invention also relates to a homogeneous population of transgenic animals whose genome comprises a heterozygous mutation of the CCN1 gene may be predisposed to AVSDs.

The transgenic animal may be an embryo. Transgenic embryos may be cultured as reviewed in the following: Tam, “Postimplantation mouse development: whole embryo culture and micro-manipulation,” Int J Dev Biol., 42:895-902 (1998); Beckman et al., “Investigations into mechanisms of amino acid supply to the rat embryo using whole-embryo culture,” Int J Dev Biol., 41:315-8 (1997); and Kane, “A review of in vitro gamete maturation and embryo culture and potential impact on future animal biotechnology,” Anim Reprod Sci., 79:171-90 (2003), the contents of which are each incorporated by reference in their entirety. Embryonic cultures of the transgenic animals may be used for the screening of modulators of AVSD.

Although the making of transgenic animals is illustrated herein with reference to transgenic mice, this is only for illustrative purpose, and is not to be construed as limiting the scope of the invention. This specific disclosure can be readily adapted by those skilled in the art to produce transgenic animals using any non-human vertebrate organism, including other mammals (e.g., rat, rabbit, sheep, cow, pig, and horse, among others) or birds (e.g., chicken). Transgenic animals of the present invention may be made using the methods described herein, as well as methods known to those of skill in the art, including the methods described in U.S. Pat. No. 6,632,979, which is incorporated by reference in its entirety.

The heterozygous mutation of the CCN1 gene may be a disruption of the CCN1 including, but not limited to, a knock-out or preferably an insertional inactivation (i.e., a “knock-in”) by introducing an identifiable marker gene such as lacZ encoding β-galactosidase. Of course, many mutant constructions are possible, including mutants resulting from the replacement of wild-type sequence by related sequences that specify variant amino acid sequences.

The present invention also relates to a gene therapy treatment for treatment of AVSDs mediated by a heterozygous mutant of CCN1 comprising the introduction of a wild type CCN1 gene into a cell.

The present invention also relates to a method of identifying a modulator of symptoms associated with AVSDs comprising contacting a transgenic mouse whose genome comprises a heterozygous mutations of the CCN1 gene with a suspected modulator and measuring a phenotype associated with AVSDs, whereby a modulator is identified by altering the phenotype in comparison to a control.

The transgenic animals and cell lines are particularly useful in screening compounds that have potential as therapeutic treatments of congenital heart disease. Screening for a useful drug involves administering the candidate drug over a range of doses to the transgenic animal, and assaying at various time points for the effect(s) of the drug on symptoms associated with congenital heart disease. Alternatively, or additionally, the drug can be administered prior to or simultaneously with exposure to an inducer of the disease, if applicable.

In addition to screening a drug for use in treating AVSDs, the transgenic animals of the present invention are also useful in designing a therapeutic regimen aimed at treating the symptoms associated with AVSDs. For example, the animal may be treated with a combination of a particular diet, exercise routine, surgery, biodevice and/or one or more compounds identified herein either prior to, simultaneously, or after the onset of symptoms associated with AVSDs. Such an overall therapy or regimen might be more effective at treating the symptoms associated with congenital heart disease than treatment with a compound alone.

The present invention also relates to a diagnostic for AVSDs. Such a diagnostic would be useful for genetic counseling, diagnosing AVSDs and the likelihood of its occurrence. Cells may be obtained from an individual or fetus, and optionally expanded, using a variety of methods known to those of skill in the art. DNA may be isolated and analyzed for mutations in the CCN1 gene using materials and methods described in U.S. Pat. No. 6,413,735. The capacity of the CCN1 nucleic acid to be expressed may be dispositive in the diagnosis of AVSDs.

Throughout this application, where publications or patents are referenced, the disclosures of these publications or patents in their entireties are hereby incorporated by reference into this application in order to more fully describe the state of the art to which this invention pertains.

The present invention has multiple aspects, illustrated by the following non-limiting examples.

### EXAMPLE 1

### CCN1 Knock-Out Mice

The initial step in preparing knock-out mice was to construct a targeting vector that contained the mouse CCN1 gene insertionally inactivated by introducing the bacterial lacZ gene encoding β-galactosidase, which facilitated screening for knock-out mice. A commercially available 129 SvJ mouse genomic DNA library (Stratagene) was screened with a CCN1 probe and Clone 61-9 was identified. Clone 61-9 phage DNA was then prepared and digested with StuI and BamHI using conventional techniques. The 6 kb fragment containing the CCN1 promoter and coding region was ligated to a blunt-ended KpnI linker, thereby attaching the linker to the Stul site. The fragment was then digested with BamHI and KpnI and inserted into BamHI, KpnI digested pBluescript KS+. The recombinant pBluescript KS+ was cut with SmaI and then ligated to an XhoI linker. Alter linker ligation, the recombinant plasmid was cut with XhoI and the XhoI fragment bearing the lacZ coding region from pSAβgal (Friedrich et al., Genes Dev. 5:1513-1532 (1991)) was inserted. The PGK-TK-blue plasmid containing a thymidine kinase gene driven by the PGK promoter (Mansour et al., Nature 336:348-352 (1988)) was cut with EcoRI and the ends were blunted with Klenow. The blunt-ended fragment was then ligated to KpnI linkers. Finally, the CCN1-βgal-neo DNA and the modified PGK-TK DNA were each cut with KpnI and ligated to generate p61 geo, the final targeting construct. Thus, p61geo contained functional βgal and neo coding regions flanked on the 5′ side by a 1.7 kb fragment containing an intact CCN1 promoter and flanked on the 3′ side by a 3.7 kb fragment containing the 3′ end of the CCN1 coding region (exons 2-5 and 3′ flanking sequence). Homologous recombination of this insert into the mouse chromosome would disrupt the CCN1 coding region and place the βgal and neo coding regions into the genome.

Cell culturing was performed according to Genome Systems instructions for mouse embryonic fibroblasts (MEFs), or as described by Li et al., Cell 69:915-926 (1992), with modifications, for J1 ES cells. Briefly, MEFs were cultured in 7.5% CO₂ in an incubator at 37° C. with DMEM (high glucose) medium (Gibco/BRL #11965-084) and 10% heat-inactivated Fetal Calf Serum (HyClone), 2 mM glutamine, 0.1 mM non-essential amino acids, and optionally with 100 U of Penicillin/Streptomycin. MEFs were isolated from mouse embryos at E14.5 and supplied at passage 2.

For feeder cells, MEFs were mitotically inactivated by exposure to 10 μg/ml Mytomycin C (Sigma) in culture medium at 37° C. (7.5% CO₂) for 2-5 hours. Cells were then washed 3 times with PBS. Mitotically inactivated MEFs were harvested with trypsin-EDTA (Gibco/BRL) and plated at about 1×10⁵/cm² with MEF medium.

J1 embryonic stem (ES) cells were cultured in DMEM (no pyruvate, high glucose formulation; Gibco/BRL# 11965-084) supplemented with 15% heat inactivated FCS (Hyclone), 2 mM glutamine (GibcoBRL), 0.1 mM non-essential amino acids (GibcoBRL), 10 mM HEPES buffer (Gibco/BRL), 55 μM β-mercaptoethanol (Gibco/BRL), and 1,000 U/ml ESGRO (leukemia inhibitory factor, LIF)(Gibco/BRL). J1 cells were routinely cultured in ES medium on a feeder layer of mitotically inactivated MEFs in a humidity saturated incubator at 37° C. in 7.5% CO₂. Normally, 1.5×10⁶ J1 cells were seeded in a 25 cm² tissue culture flask and the medium was changed every day. Cell cultures were divided 2 days after seeding, usually when the flask was about 80% confluent. To dissociate ES cells, cells were washed twice with PBS (Ca- and Mg-free) and trypsinized with Trypsin/EDTA at 37° C. for 4 minutes. Cells were then detached, mixed with trypsin/EDTA thoroughly, and incubated for an additional 4 minutes. The cell suspension was then pipetted several (20-30) times to break up the cell clumps. A complete dissociation of cells was checked microscopically. ES cells were frozen with ES medium having 10% FCS and 10% DMSO (Sigma) at about 4-5×10⁶ cells/ml, with 0.5 ml/tube. Frozen cells were stored at −70° C. overnight and transferred into liquid nitrogen the next day. Frozen cells were quickly thawed in a 37° C. water bath, pelleted in 5 ml ES medium to remove DMSO, and plated in 25 cm² flasks with fresh MEF feeder cells.

To transfect mouse cells with a transgene, the p61 geo targeting construct was linearized by NotI digestion, suspended in PBS at 1 μg/ml, and introduced into J1 ES cells by electroporation. Rapidly growing (subconfluent, medium newly refreshed) J1 ES cells were trypsinized, counted, washed and resuspended in the electroporation buffer containing 20 mM HEPES, pH 7.0, 137 mM NaCl, 5 mM KCl, 6 mM D-glucose, and 0.7 mM NaZHPO₄, at 1×10⁷ cells/ml. Linearized DNA was added to the cell suspension at 45 μg/ml, mixed, and incubated at room temperature for 5 minutes. An 0.8 ml aliquot of cell-DNA mix was then transferred to a cuvette and subjected to electroporation with a BioRad Gene Pulser using a single pulse at 800 V, 3 μF. Cells were left in the buffer for 10 minutes at room temperature, and then plated at 4×10⁶ cells/100 mm plate with neomycin-resistant MEF feeder cells. Cells were then cultured under standard conditions without drug selection. After 24 hours, selection medium containing ES medium supplemented with 400 μg/ml (total) 6418 (Gibco/BR.L) and 2 μM Ganciclovir (Roche) was substituted. Selection medium was refreshed daily. Individual colonies were placed in microtiter wells and cells were dissociated with 25 μl 0.25% trypsin-EDTA/well on ice and subsequently incubated in a humidified incubator at 37° C. with 7.5 CO₂, for 10 minutes. Cell suspensions were then mixed with 25 μl ES medium and pipetted up and down 10 times to break up clumps of cells. The entire contents of each well were then transferred to a well in a 96-well flat-bottom dish with 150 μl of ES medium in each well and grown using conventional culturing techniques for 2 days.

Confluent ES cell clones were washed and treated with lysis buffer (10 mM Tris (pH 7.7), 10 mM NaCl, 0.5% (w/v) sarcosyl, and 1 mg/ml proteinase K) in a humid atmosphere at 60° C. overnight. After lysis, a mixture of NaCl and ethanol (150 μl of 5 M NaCl in 10 ml of cold absolute ethanol) was added (100 μl/well) and genomic DNA was isolated. The genomic DNA of each ES cell clone was digested with EcoRI (30 μl/well) and subjected to Southern blot assay.

Southern blotting was preformed as described in “Current Protocols in Molecular Biology” (Ausuhel et al., [1999]). Briefly, EcoRI fragments of genomic DNA were fractionated by electrophoresis through 0.8% agarose gels and blotted onto nylon membranes (Bio-Rad) by downward capillary transfer with alkaline buffer (0.4 M NaOH). The probes, a RumHI-EcoRI fragment 3′ to the long arm of the targeting construct (p61 geo) or the neo coding region sequences, were prepared by random primer labeling (Prim-it 118, Stratagene) using [(α-³²P] dCTP (NEN). Membranes were prehybridixed in hybridization buffer (7% SDS, 0.5 M NaHPO₄ (pH 7.0), and 1 mM EDTA) at 65° C. for 15 minutes in a rolling bottle. Fresh hybridization buffer was added with the probe and membranes were hybridized for 18 hours. Hybridized membranes were briefly rinsed in 5% SDS, 40 mM NaHPO₄ (pH 7.0), 1 mM EDTA and then washed for 45 minutes at 65° C. with fresh wash solution. The wash solution was replaced with 1% SDS, 40 mM NaHPO₄ (pH 7.0), 1 mM EDTA and washed twice for 45 minutes at 65° C. with fresh solution. Alter washing, membranes were exposed to a screen, which was then scanned using a Phosphorlmager® (Molecular Dynamics). Blots were routinely stripped and re-probed with the control neo probe to ensure that random integration had not occurred, using conventional techniques.

Results of the Southern analysis showed that the genomic DNA of 14 colonies (231 colonies examined) contained a mutant CCN1 allele in a location consistent with integration via homologous recombination. The sizes of the detected fragments were 6.4 kb for the wild-type CCN1 allele and 7.4 kb for the mutant allele with the cyr61 probe; no band for the wild-type CCN1 allele and a 7.4 kb band for the mutant allele with the neo probe.

Genotyping was also done by PCR using a RoboCycler® (Stratagene). Primers were designed to amplify a 2.1 kb DNA fragment from mutant alleles. The PCR product covers the 5′-flank of the short arm of the targeting construct through to the sequence of lacZ (β-gal) within the targeting construct. The upper PCR primer sequence was 5′-CACAACAGAAGCCAGGAACC-3′ (SEQ ID NO:1) and the lower PCR primer sequence was 5′-GAGGGGACGACGACAGTATC-3′ (SEQ ID NO:2). PCR reaction conditions were 95° C. for 40 seconds, 63° C. for 40 seconds, and 68° C. for one minute, for 35 cycles.

For genotyping mouse tails or embryo tissues, two sets of primers were included in the same PCR reaction to amplify both wild-type and mutant alleles. A single upper PCR primer (b) was used, which had the sequence 5′-CAACGGAGCCAGGGGAGGTG-3′ (SEQ ID NO:3). The lower PCR primer for amplifying the wild-type allele, lower wt primer, had the sequence 5′-CGGCGACACAGAACCAACAA-3′ (SEQ ID NO:4) and would amplify a fragment of 388 bp. The lower PCR primer for amplifying the mutant allele was the lower mutant primer and had the sequence 5′-GAGGGGACGACGACAGTATC-3′ (SEQ ID NO:5); a 600 bp fragment was amplified from mutant alleles. Reaction conditions were: 95° C. for one minute, 63° C. for one minute, and 72° C. for one minute, for 30 cycles.

PCR amplification of mutant alleles of CCN1 using the mutant-specific primers produced a fragment of 2.1 kb and attempts to amplify the wild-type allele with those primers failed to produce a detectably amplified fragment, in agreement with expectations. Southern analyses identified a 7.4 kb band (mutant allele) and a 6.4 kb band (wild type) in heterozygous mutants; only the 6.4 kb band was detected when probing wild-type DNAs. Both the PCR data and the Southern data indicate that mutant CCN1 alleles were introduced into the mouse genome in a manner consistent with homologous recombination.

The selected ES cell clones were then expanded for micro-injection into E3.5 blastocysts from C57BL/6J mice. Embryo manipulations were carried out as described by Koblizek et al., Curr. Biol. 8:529-532 (1998) and Suri et al., Science 282:468-471 (1998), with modifications. Briefly, the J1 ES cell clones were harvested and dissociated with trypsin-EDTA. The cells were resuspended in CO₂-independent medium (Gibco-BRL) with 10% FBS and kept on ice. About 15-20 ES cells were injected into each blastocyst from C57BL/6J (Jackson Labs). Injected blastocysts were cultured for 1-2 hours prior to transfer into the uterine horns of pseudopregnant foster mothers (CD-1, Harlan). Chimeras were identified by coat color. Male chimeras with a high percentage of agouti coat color were caged with C57BL/6J females to test germ-line transmission of the ES-cell genotype. F₁ offspring carrying the targeted (i.e., mutant) allele were then back-crossed with C57BL/6J females for a few rounds to establish an inbred C57BL genetic background. In addition, a mutant mouse line having the inbred 129SvJ genetic background was obtained by mating germ-line chimera males with 129SvJ females.

Five ES cell clones were injected and generated chimeric offspring with ES cell contributions ranging from 30%-100%, as judged by the proportion of agouti coat color. Four and two chimeric males derived from ES cell clones 4B7 and 2A11, respectively, efficiently transmit the targeted allele through the germline. The CCN1 heterozygous mutant mice appeared healthy and fertile. The 4B7 chimeric line was either bred to 129SvJ mice to maintain the targeted allele in a SvJ129 genetic background, or back-crossed with C57BL/6J mice to transfer the mutation into the C57BL/6J background. The 2A11 targeted line was maintained in the 129SvJ genetic background. Similar phenotypes were exhibited by the 4B7₁₂₉, 4B7_{C57BL}, and 2A11 mouse lines.

Among the offspring from intercrosses of cyr61^{+/-} mice that were examined, 141 were CCN1^{+/+}, 225 were CCN1^{+/-}, and no homozygous CCN1^{-/-} mice were observed at this age, except that 10 CCN1^{-/-} pups were born alive and died within 24 hours of birth. Based on Mendelian ratios, the majority (>90%) of the CCN1^{-/-} animals should have died before birth. Thus, staged prenatal fetuses were examined by PCR, as described above. Starting from E10.5, the numbers of homozygous mutant embryos were found to be less than expected based on a Mendelian ratio, which might have been due to resorption of homozygous mutant embryos. However, most (80%) of the E10.5 CCN1^{-/-} embryos appeared normal compared to littermates. At this stage (E10.5), the failure of chorioallantoic fusion was found in some embryos and this phenotype resulted in early embryonic lethality. The allantois of this type of embryo appeared ball-shaped and often was filled with blood. While no other defects were specifically identified, hemorrhage began to appear in a few of the CCN1-null embryos.

At E11.5, about 50% of CCN1^{-/-} embryos were indistinguishable from wild-type or heterozygous mutant littermates by appearance. By E11.5, embryos lacking a chorioallantoic fusion were consistently deteriorated. Increasing numbers and severity of hemorrhage were also observed in CCN1-null embryos. Hemorrhages occurred in different areas, including the placenta, intra-uterus, intra-amnion, embryo body trunks, body sides, and head. At this stage, placental defects were also found in some null mutant embryos. The placentae associated with these embryos showed a sub-standard vasculature network. Unlike the early lethality associated with the failure of chorioallantoic fusion, embryos with placental defects typically lived and developed normally.

At E12.5, CCN1^{-/-} embryos still presented three phenotypes: 1) unaffected, 2) alive with hemorrhage and/or placental defects, and 3) deteriorated, though with the proportion of categories changed from earlier stages. About 30% of the CCN1-null embryos remained unaffected at this stage. About 50% of the null mutant embryos showed signs of hemorrhage and/or placental defects and 20% of this type of embryo did not survive the vascular or the placental defects. About 20% of CCN1^{-/-} embryos did not have a chorioallantoic fusion and died at much earlier stages, as judged by the under-development of defective embryos.

By E13.5, none of the CCN1^{-/-} embryos that had shown hemorrhage, placental defects, or failure of chorioallantoic fusion were alive, although about 30% of the total CCN1-deficient embryos showed no apparent phenotype. Embryos examined at later stages (>E14.5) showed the same phenotypic pattern and the same proportion for each type of defect, but with increasing severity.

Additional investigation, at the cellular and sub-cellular levels, was performed using the following techniques. MEF cells were harvested as described by Hogan et al., Manipulating the Mouse Embryo-A Laboratory Manual (1994). Briefly, E11.5 embryos from crosses of two heterozygous CCN1-targeted parents were dissected in DMEM without serum. The limbs, internal organs, and brain were removed. Embryo carcasses were then minced with a razor blade and dissociated with trypsin/EDTA at 37° C. with rotation for 10 minutes. Half of the dissociation buffer was then added to an equal volume of DMEM plus 10% FBS. Dissociation and collection steps were repeated five times. Collected cells were expanded and split at a 1:10 ratio to select the proliferating fibroblast cells.

To prepare total cell lysates, a 100 mm plate of MEF cells was cultured to near confluency. Cells were activated with fresh medium containing 10% serum and incubated at 37° C. for 1.5 hours before being harvested. Cells were then washed and centrifuged using conventional procedures. The cell pellets were resuspended in 100 μl RIPA buffer (0.5% sodium deoxycholate, 0.1% SDS, 1% Nonidet P-40,50 mM Tris-Cl, pH 8.0, 150 mM NaCl, aprotinin 0.2 units/ml, and 1 mM PMSF) and put on ice for 10 minutes to lyse the cells. The cell suspension was centrifuged and the supernatant (cell lysate) was stored at −70° C. for further analysis. One third of the supernatant was subjected to Western blot analysis using a TrpE-mCCN1 polyclonal anti-serum.

To confirm that homozygous CCN1^{-/-} animals did not express CCN1, MEF cells were prepared from E1.5 embryos resulting from intercrosses of two CCN1^{+/-} parents. Cell lysates were collected from serum-stimulated MEFs of different genotypes and were subjected to Western blot analyses using anti-CCN1 antiserum (trpE-mCCN1). The Western blot demonstrated that the CCN1 protein level was not detectable in KO (knockout) MEF cells, while heterozygous CCN1^{+/-} cells expressed the CCN1 protein at high levels under the same culture conditions and serum stimulation. The lack of expression of CCN1 in CCN1^{-/-} animals was further confirmed by Northern blot analyses, in which CCN1 mRNA was not detectable in serum-induced KO MEF cells. Thus, the null mutation of CCN1^{-/-} has been confirmed as eliminating CCN1 expression at both the mRNA and protein levels.

Defects in placental development, a major cause of embryonic death in CCN1^{-/-} mice, were further analyzed. Histological analyses of mouse placentae generally followed Suri et al., (1998). Briefly, placentae from E12.5 embryos were dissected in cold PBS and fixed with 4% paraformaldehyde in 0.1 M phosphate buffer (PB) at 4° C. for overnight. Fixed placentae were then dehydrated through increasing concentrations of alcohol (50%, 75%, 90%, 95%, and 100%) two times. Dehydrated tissue was then cleared with Hemo-De (a xylene alternative), 1:1 ethanol/Hemo-De (Fisher), and 100% Hemo-De, and the clearing process was repeated. Cleared tissues were then equilibrated in a 1:1 mixture of paraffin:Hemo-De at 60° C. for one hour in a vacuum oven and the process was repeated. Tissues were embedded in paraffin with Histoembedder (Leica). The paraffin-embedded placentae were cut into 10 μm slices with a microtome (Leica). Finally, tissue sections were subjected to Harris' Hematoxylin and Eosin staining (Asahara et al. Circ. Res. 83:233-240 [1998]).

Placentae for immunohistochemical staining were dissected in cold PBS and fixed in 4% paraformaldehyde at 4° C. overnight. Fixed tissue was transferred to 30% sucrose in PBS at 4° C. overnight. Placentae were then embedded in O.C.T. (polyvinyl alcohol, carbowax solution) on dry ice. Frozen blocks were stored at −70° C. or cut into 7 μm sections with a cryotome (Leica). Immunohistochemical staining was done as recommended by the manufacturer (Zymed). Briefly, frozen sections were post-fixed with 100% acetone at 4° C. for 10 minutes. Endogenous peroxidase was blocked with Peroxo-Block (Zymed). Sections were incubated with a 1:250 dilution of biotinylated rat anti-mouse PECAM-1 (i.e., platelet endothelial cell adhesion molecule-1) monoclonal antibody MEC 13.3 (Pharmingen) at 4° C. overnight. A Histomouse-SP kit with Horse Radish Peroxidase (Zymed) was used to detect PECAM-1 signals.

The results of histological and immunohistochemical analyses showed that CCN1-null placentae contained a limited number of embryonic blood cells and were largely occupied by maternal blood sinuses. Abnormally compact trophoblastic regions were also observed. PECAM-1 staining demonstrated the highly-vascularized labyrinthine zone in a heterozygous mutant placenta. Under higher magnification, flows of fetal blood cells within the PECAM-1 stained vessels were identified. Consistent with the variation in phenotypes among the CCN1-deficient embryos, the staining of placentae from numerous CCN1^{-/-} embryos also reflected placental defects to various degrees. Nonetheless, the placental defects observed with PECAM-1 staining can be classified into two groups, groups I and II. Group I of type II (type I-embryos with complete failure of chorioallantoic fusion not surviving E10.5; type II-embryos with partially defective chorioallantoic fusion surviving through about E13.5) exhibits a set of placental defects that is characterized by the virtual absence of embryonic vessels, the presence of condensed trophoblasts, and the presence of a compressed labyrinthine zone. A higher magnification view confirms that no vessels developed in the labyrinthine with placental defects of this kind. Placentae with a group II defect showed fair amounts of PECAM-1-positive staining and condensed capillary structures. However, the PECAM-1-stained vessel-like structures were degenerated and collapsed, with no fetal blood cells inside.

Thus, the lack of CCN1 causes two types of placental defects. In type I, the failure of chorioallantoic fusion results in the loss of physical connection between the embryo and the placenta. In type II placental defects, the physical connection is established by successful chorioallantoic fusion. However, the embryonic vessels only reach to the surface of the placenta or, with successful penetration through the chorionic plate, develop an immature non-functional vascular structure in the labyrinthine zone.

X-gal staining was also used to assess embryonic development in various xyr61 backgrounds. (The targeting DNA, p61geo, was designed to knock out the CCN1 gene and also to “knock in” a β-gal gene as a marker to reflect the expression of CCN1). X-gal (i.e., 5-Bromo-4-chloro-3-indolyl-β-D-galactopyranoside) staining for β-galactosidase expression was performed on heterozygous CCN1^{+/-} embryos staged from E9.5 to E11.5. The staining was done as described (Surfet al., [1998]). Staged embryos were fixed in a 0.2% paraformaldehyde solution at 4° C. overnight. Fixed tissue was incubated in 30% sucrose in PBS plus 2 mM MgCl₂ at 4° C. overnight. Tissue was then embedded in OCT on dry ice and cut with a cryotome into 7 μm sections. Frozen tissue sections were post-fixed in 0.2% paraformaldehyde and stained with X-gal (1 mg/ml) at 37° C. for 3 hours in the dark. Slides were counter-stained with 1% Orange G. Stained slides were then serially dehydrated through increasing concentrations of methanol, cleared with Hemo-De, and slides were mounted.

X-gal staining of the E9.5 embryos, including the extra-embryonic tissues, showed β-galactosidase expression, driven by the CCN1 promoter, at the tip of the allantois adjacent to the chorion in the chorioallantoic placenta. The staining of more advanced E10.5 embryos illustrated that large vessels branching from the allantoic vessels were developed in the chorionic plate and could easily be identified in the endothelial lining using X-gal. Further developed E11.5 placenta showed the same expression pattern as E10.5 embryos. While the staining was highly associated with the endothelium of the umbilical and chorionic vessels, no detectable staining in the labyrinthine zone, where a microvasculature network was developing, was seen at E11.5. The presence of CCN1 in the allantois at, and proximal to, the fusion surface with the chorion, and in the umbilical and chorionic vessels, further supports the important role of CCN1 in angiogenesis. CCN1 was involved in chorioallantoic fusion and was critical for proper angiogenic development as placentation progressed. Moreover, a staining of the E11.5 embryo confirmed that CCN1 was expressed in the paired dorsal aortae and the major arteries branching from the heart, which is consistent with the hemorrhaging seen in CCN1-null mutants.

Also apparent from the preceding description is another aspect of the invention, which is drawn to a mammalian cell comprising a CCN1 mutation selected from the group consisting of an insertional inactivation of a CCN1 allele and a deletion of a portion of a CCN1 allele. The mammalian cell is preferably a human cell and the mutation is either heterozygous or homozygous. The mutation, resulting from insertional inactivation or deletion, is either in the coding region or a flanking region essential for expression such as a 5′ promoter region. Cells are also found associated with non-human animals.

### Example 2

### Cardiovascular Defects in Heterozygous Transgenic Mice

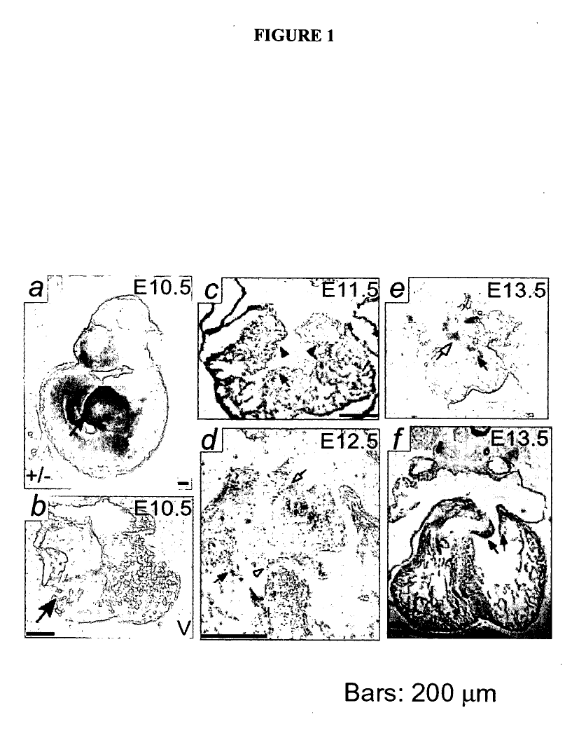

In creating the CCN1 null mutation, a lacZ reporter gene was incorporated to be driven by the CCN1 promoter after homologous recombination. The expression of CCN1, represented by X-gal staining, is predominant at the heart at E10.5 (FIG. 1a). High level of expression was found in truncus arteriosus (FIG. 1b), which will later divide to form the aorta and pulmonary trunk. The expression was also seen around the atrioventricular canal at later stages (FIGS. 1c, d, e, f).

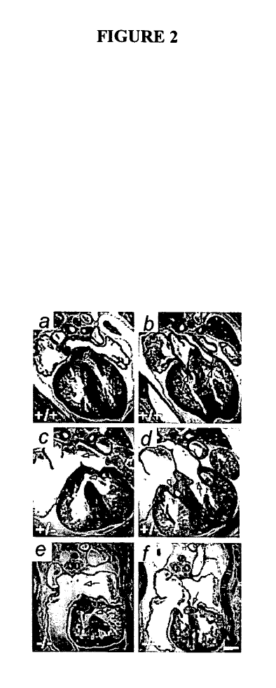

Based on its expression pattern, biological activities and mapping to the AVSD critical region, the cardiac system of CCN1^{-/-} embryos was carefully examined. Surprisingly, cardiac abnormalities were identified in both CCN1^{+/-} and CCN1^{-/-} embryos by E13.5 indicating an autosomol dominant in human AVSD mutations. At this stage, the four-chamber structure centered by the atrioventricular (AV) endocardial cushion tissue of the heart has developed by formation of the atrial and ventricular septa, and the AV valves (FIG. 2a, 2b). The formation of the valve leaflets and septa requires proper development of the AV cushion. The cushion arises from the outgrowth of the mesenchyme, which is transformed from the endothelial cells lining the AV canal and migrate into regional swellings of the cardiac jelly composed of ECM proteins {Eisenberg & Markwald 1995 ID: 283}.

Histological analysis of the hearts from E13.5 and E14.5 embryos demonstrated that ˜65% of CCN1^{+/-} embryos (n=27) possessed cardiac defects of different severity while the wild type littermates developed normally. Four (4/27) of the heterozygous embryos displayed complete AVSDs. The most common defect detected at this stage was interventricular septal defect (VSD)(15/27). The formation of the interventricular septum (IVS) involves the expansion of the muscular component of the septum from ventricular wall and the extension of the membranous component composed of the mesenchymes from the AV cushion tissue. The defective hearts from CCN1^{+/-} embryos possess normal expansion of muscular component whose upper borders were well positioned proximal to the AV cushion, however, the cushion mesenchymes fail to migrate and establish contact with the muscular component (FIG. 2c). The expression of CCN1 is at the surfaces of both endocardial cushion tissue and IVS at E11.5 (FIG. 1c). During the onset of closing IVS at E12.5, CCN1 staining is highlighted at the junction between the septum muscular component and the membranous mesenchyme (FIG. 1d). As an extracellular matrix molecule and a ligand for integrins, CCN1 can reduce the expression of collagen and elevate the level of collagenases {Chen, MO, et al. 2001 368/id}, which strongly suggests its role on tissue remodeling at this area to allow different cell types from each components to amalgamate together.

Three out of the 27 heterozygous embryos examined showed isolated cleft mitral valves (FIG. 2d, arrow). The formation of AV valve leaflets initiates with the transformation of the endocardium endothelial cells to the prevalvular mesenchyme. The mesenchyme proliferates and differentiates into valvular tissue. The differentiated mesenchyme then induce myocardialization by luring the invasion of proximal myocardial cells {Eisenberg & Markwald 1995 ID: 283}. In the affected heterozygous CCN1^{+/-} embryos, the AV cushion tissue appeared to be underdeveloped with limited leaflet structure into ventricular lumen (FIG. 2d) comparing to wild-type littermates at E14.5 (FIG. 2b). The expression of CCN1 was identified at the developing AV valvular structure (FIG. 1f). Comparing the AV cushion and valve development among CCN1^{-/-}, CCN1^{+/-} and wild-type embryos, the cushion and valvular structures of the CCN1^{-/-} embryos clearly were underdeveloped and appeared smaller in size (FIGS. 2e,f). Although not a mitogen by itself, CCN1 has been shown to be able to potentiate mitogenic activity of other growth factors, such as bFGF {Kireeva, MO, et al. 1996 ID: 199}. Further studies are undergoing to clarify whether CCN1 is involved specifically in the proliferation, or also in the later differentiation of mesenchymal cells and the muscularization of the leaflets.

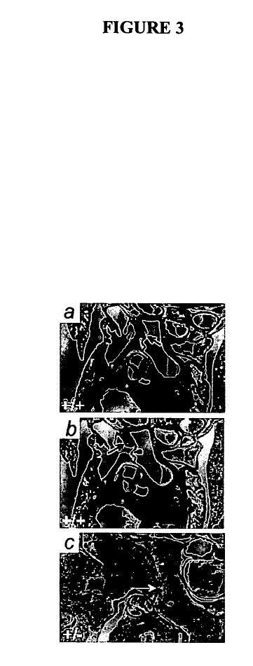

The septation of common atrial chamber starts at E10.5 with the formation of a flimsy structure, septum primum, from the middle part of the chamber wall towards the AV cushion tissue. As septum primum approaching AV cushion, the communicating space between two sides of the atrium, termed as “ostium primum,” is gradually diminished and eventually closed by fusion of the septum primum and the AV cushion tissue. The interatrial communication will be replaced by newly formed ostium secundum (FIG. 3a). The second component of atrial septum, septum secundum, forms a thicker and stiffer membrane at later stage (E13.5) and, in contrast to the septum primum, the septum secundum never reaches the AV cushion (FIG. 3b). The septum primum and septum secundum will attach to each other and completely block the communicating window between two sides of atrium chambers shortly after birth {Kaufinan & Bard 1999 ID: 286} (FIGS. 3a,b). By E14.5, under-development of the atrial septum can be detected in CCN1^{+/-} (FIG. 2c) and CCN1^{-/-} (FIG. 2d).

The postnatal atrial septation of CCN1^{+/-} pups and wild-type littermates was examined by histological analysis. The septum secundum of CCN1^{+/-} appeared to be normal, however, the shunt under the lower border of the septum secundum was not blocked by the septum primum (FIG. 3c), while the wild-type littermates demonstrated complete septation (FIGS. 3a,b). This septation deficiency results from the persistence of ostium primum due to the failure of fusion between the septum primum and AV cushion tissue. The expression of CCN1 was detected at the AV cushion tissue in the area where the fusion between septum primum and cushion tissue occurs (FIG. 1d). Similar to its role with IVS, CCN1 may facilitate the establishment of the contact and the fusion of the septum primum with AV cushion.

The variable cardiac defects in heterozygous CCN1^{+/-} mice are acquired, likely, due to haploinsufficiency as most of the human CHDs. The CCN1-null mutation resulted in more severe defects (FIGS. 2e,f) with 100% penetrance. The more severe cardiac failure found in CCN1^{-/-} likely account for the perinatal death.

1. A transgenic mouse in contact with a suspected modulator of effects associated with congenital heart disease, wherein the genome of said mouse comprises a heterozygous disruption of the CCN1 gene.

2. The mouse of claim 1, wherein said mouse is predisposed to atrioventricular septal defects.

3. The mouse of claim 1, wherein said mouse has atrioventricular septal defects.

4. The mouse of any one of claims 1-3, wherein said mouse is an embryo.

5. A homogeneous population of transgenic mice whose genome comprises a heterozygous disruption of the CCN1 gene, wherein said mice are predisposed to have atrioventricular septal defects.

6. The mice of claim 5, wherein said mice have atrioventricular septal defects.

7. The mice of any one of claims 5-6, wherein said mice are embryos.

8. The mice of claim 7, wherein one or more of said mice are in contact with a suspected modulator of effects associated with congenital heart disease.

9. The mice of any one of claims 5-6, wherein one or more of said mice are in contact with a suspected modulator of effects associated with congenital heart disease.

10. A method of producing a mouse with atrioventricular septal defects, comprising:
(a) producing a transgenic mouse whose genome comprises a heterozygous disruption of the CCN1 gene;
(b) testing the transgenic mouse for the presence of a phenotype associated with atrioventricular septal defects; and
(c) isolating a transgenic mouse that has a phenotype associated with atrioventricular septal defects.

11. A mouse produced by the method of claim 10.

12. The mouse of claim 11, wherein said mouse is an embryo.

13. A method of isolating a mouse with atrioventricular septal defects, comprising,
(a) testing a transgenic mouse whose genome comprises a heterozygous disruption of the CCN1 gene for the presence of a phenotype associated with atrioventricular septal defects; and
(b) isolating a transgenic mouse that has a phenotype associated with atrioventricular septal defects.

14. A mouse isolated by the method of claim 13.

15. The mouse of claim 14, wherein said mouse is an embryo.

16. A method of identifying a mouse with atrioventricular septal defects, comprising testing a transgenic mouse whose genome comprises a heterozygous disruption of the CCN1 gene for the presence of a phenotype associated with atrioventricular septal defects.

17. A mouse produced by the method of claim 16.

18. The mouse of claim 17, wherein said mouse is an embryo.

19. A method of identifying a modulator of symptoms associated with atrioventricular septal defects, comprising:
(a) contacting a transgenic mouse whose genome comprises a heterozygous disruption of the CCN1 gene with a suspected modulator;
(b) measuring a phenotype associated with atrioventricular septal defects, whereby a modulator is identified by altering the phenotype in comparison to a control.

20. The method of claim 19, wherein said mouse is an embryo.

21. A method of identifying an animal that is predisposed to atrioventricular septal defects, comprising detecting the presence of an alteration in one or more alleles of the CCN1 gene in a sample comprising DNA isolated from said animal.
